(19) United States
(12) Patent Application Publication
(10) Pub. No.: US 2026/0144626 A1
(43) Pub. Date: May 28, 2026

(54) ARTIFICIAL BLADDER

(71) Applicant: Ernesto Andrade, Miami Beach, FL (US)

(72) Inventor: Ernesto Andrade, Miami Beach, FL (US)

(21) Appl. No.: 19/451,438

(22) Filed: Jan. 16, 2026

Related U.S. Application Data
(63) Continuation-in-part of application No. 19/290,775, filed on Aug. 5, 2025.
Application No. 19/290,775 is a continuation of application No. 17/659,245, filed on Apr. 14, 2022, now Pat. No. 12,383,392.
(60) Provisional application No. 63/201,141, filed on Apr. 14, 2021.

(51) Int. Cl. A61F 2/04; A61F 2/48

(52) U.S. Cl. CPC A61F 2/042; A61F 2/48; A61F 2220/0075

(57) ABSTRACT

An implantable medical device for use as an artificial urinary bladder can include a collection container configured to be placed in a patient's abdominal cavity and a pump configured to be subcutaneously placed outside the abdominal cavity to allow the patient to perceive the need for urination and controls timing of the urination. Flexible tubing can connect the patient's ureters to the collection container through anti-reflux valves preventing urine from flowing back to kidneys, connect the collection container to the pump through a unidirectional connection valve, and connect the pump to the patient's urethra through a unidirectional urethral valve to allow for urination through the patient's natural urethra.

### CROSS REFERENCE TO RELATED APPLICATION

This application is a continuation-in-part of co-pending U.S. patent application Ser. No. 19/290,775, entitled “ARTIFICIAL BLADDER”, filed on Aug. 5, 2025, which is a continuation of U.S. patent application Ser. No. 17/659,245, entitled “ARTIFICIAL BLADDER”, filed Apr. 14, 2022, now issued as U.S. Pat. No. 12,383,392, which claims the benefit of priority under 35 U.S.C. § 119(e) of U.S. Provisional Patent Application Ser. No. 63/201,141, entitled “ARTIFICIAL BLADDER”, filed on Apr. 14, 2021, which are herein incorporated by reference in their entirety.

### TECHNICAL FIELD

This document relates generally to implantable medical devices and more particularly, but not by way of limitation, to an implantable medical device that can be used as an artificial bladder for collecting and discharging urinary fluids.

### BACKGROUND

The urinary bladder (referred to as “urinary bladder” or “bladder” herein) is a hollow muscle-membranous organ that is part of a person's urinary tract. The bladder receives urine from the ureters, stores the urine, and expels the urine from the body of the person through the urethra during urination. The bladder has a spherical shape. In its posterior-superior portion, the bladder connects to the ureters through which the urine is received from the kidneys. In its inferior-medial portion, the bladder terminates in the urethra, which terminates at the urinary meatus. After entering the bladder, the urine accumulates to level that is sensed by pressure corpuscles, which send signals to the brain to initiate urination. Urination is a complex act during which the detrusor muscle contracts while the urethral sphincter relaxes.

Surgical removal of the bladder, a procedure known as radical cystectomy, may become necessary due to various reasons such as certain tumors and trauma. One example of such reasons includes bladder cancer, which is one of the most common types of cancer worldwide and also known as one of the leading causes of death. This necessities the surgical removal of the bladder for many patients. Once a patient's bladder is removed, it is necessary to restore the function previously performed by the patient's bladder, including allowing the urine to accumulate and then flow and discharge at regular intervals out of the patient's body. Currently there is surgical procedure and/or artificial device that can replace the bladder with its full natural functions.

An example of a surgical technique for restoring urinary functions after bladder removal includes harvesting a portion of the patient's intestine, adhering it to the ureters, and subsequently generating a urinary stoma across the abdominal wall. An externally carried urine collection bag is attached to the body to collect the urine through the stoma. There are two ways to manage this technique. One way includes a surgery that takes a portion of the intestine and isolates it from its full length. The remaining portions of the intestine are joined together so that the intestine continues its function. One end of “borrowed” portion of the intestine is closed. The other end is removed from the abdominal cavity through muscles and skin and to form the stoma (artificial opening) on the surface in the patient's abdominal area. The ureters are attached to this “borrowed” portion of the intestine to allow the urine to accumulate in and flow through it to the stoma. A bag is attached to the skin in the abdominal area to cover the stoma to collect the urine. This procedure, known as ureterocutaneostomy, does not restore the natural functions performed by the patient's bladder. For example, the urine does not accumulate properly within the body and is not expelled through the regular duct, the urethra. The other way is substantially the same, except that instead of forming the stoma on the skin, a larger sac is created to form a urinary storage bag. Thus, the patient has to permanently perform catheterization through the urethra to remove the urine.

Another example of a surgical technique for restoring urinary functions after bladder removal includes a surgery for harvesting a portion of the intestine and connecting it to the ureters and the urethra to create a neobladder. To create the neobladder, the patient's bladder (e.g., a cancerous or severely traumatized bladder) is removed by a procedure known as a cystectomy, either through a traditional abdominal incision or with a robot-assisted laparoscopic approach (robotic surgery). A segment of the small intestine, a portions of the colon, or a combination of both is remodeled to form a sphere, which becomes the neobladder. The neobladder is placed in the patient's body in the space that was occupied by the original bladder. The neobladder is attached to the ureters so that the urine can drain from the kidneys into the neobladder. The other end of the neobladder joins the urethra. This technique allows the patient to maintain urine control with a functional bladder capable of storing urine without the need for external device (e.g., a bag). However, potential complications can occur with the neobladder construction, such as bleeding, blood clots, infection, urinary leakage, urinary retention, electrolyte imbalances, vitamin B-12 deficiency, incontinence, and cancer in the intestine.

In addition to the great technical diffculty of the procedure, the neobladder is associated with a high morbidity with subsequent complications and a non-negligible mortality rate. Complications have been specifically detected over the years in patients who have undergone the neobladder construction surgery, related to metabolism and liver medications, vitamin deficiency, electrolyte disorders, bone diseases, cancers, and related problems with the presence of an ostomy, such as bleeding, stenosis, and hernia. Furthermore, following the procedure, serious incontinence problems are common, forcing the patient to wear diapers for incontinent adults and hence experiencing physical and psychological discomfort. There are also other types of neobladders known as heterotopic, such as the intestinal neobladder connected to the navel, emptied by the patient through a catheter. This type of neobladder also has drawbacks related to the diffculty of implantation and infections that can cause kidney failure.

Therefore, at this time, patients undergoing bladder removal have the possibility of experiencing an unsatisfactory quality of life, which also implies a significant economic cost for the health system. All external urinary components, such as bags, as well as internal components, such as neobladders, involve substantial costs for regular maintenance, which may include multiple controls and replacements performed by specialized personnel.

### SUMMARY

An implantable medical device for use as an artificial urinary bladder can include a collection container configured to be placed in a patient's abdominal cavity and a pump configured to be subcutaneously placed outside the abdominal cavity to allow the patient to perceive the need for urination and controls timing of the urination. Flexible tubing can connect the patient's ureters to the collection container through anti-reflux valves preventing urine from flowing back to kidneys, connect the collection container to the pump through a unidirectional connection valve, and connect the pump to the patient's urethra through a unidirectional urethral valve to allow for urination through the patient's natural urethra.

An example of an implantable medical device for collecting and discharging urine from a body of a patient is provided. The body has kidneys, ureters, a urethra, an abdominal cavity, abdominal muscles having a fascia, and a pelvic floor. The implantable medical device can include: first and second ureteral tubes configured to be connected to the ureters to receive the urine from the kidneys through the ureters; a collection container coupled to the first and second ureteral tubes and configured to be placed inside the abdominal cavity and to receive the urine from the first and second ureteral tubes; first and second anti-reflux valves positioned in the collection container and respectively coupled to first and second ureteral tubes, the first and second anti-reflux valves configured to prevent urine from flowing from the collection container to the kidneys; a pump configured to be placed outside of the abdominal cavity; a connection tube connected between the collection container and the pump to provide for fluid communication from the collection container to the pump; a unidirectional connection valve coupled to the connection tube and configured to allow the urine to flow from the collection container to the pump; a urethral tube connected to the pump and configured to be connected to the urethra; and a unidirectional urethral valve coupled to the urethral tube and configured to allow the urine to flow from the pump to the urethra.

An example of a method for replacing functions of a urinary bladder using an implantable medical device placed in a body of a patient is also provided. The body has kidneys, ureters, a urethra, an abdominal cavity, and a pelvic floor. The method can include: collecting urine from kidneys of the body using a collection container placed within the abdominal cavity through first and second ureteral tubes connected between the ureters and the collection container; preventing the collected urine from flowing back to the kidneys using first and second anti-reflux valves positioned in the collection container and coupled to the first and second ureteral tubes; allowing the urine to flow from the collection container to a pump subcutaneously placed outside of the abdominal cavity through a connection tube connected between the collection container and the pump when the pump is not pressed; and allowing the urine to flow from the pump to the urethra through a urethral tube connected between the pump and the urethra when the pump is pressed.

Another example of an implantable medical device configured to be used as an artificial urinary bladder in a body is provided. The body has kidneys, ureters, a urethra, an abdominal cavity, and a pelvic floor. The implantable medical device can include: first means for being placed within the abdominal cavity and collecting urine produced by the kidneys; anti-reflux means for preventing the collected urine from flowing back to the kidneys; and second means for being placed subcutaneously outside of the abdominal cavity, receiving the collected urine, and expelling the received urine from the body when being pressed.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

1 The drawings illustrate generally, by way of example, various embodiments discussed in the present document. The drawings are for illustrative purposes only and may not be to scale.

FIG. 1 illustrates an embodiment of an implantable medical device and portions of an environment in which the implantable medical device is used as an artificial bladder.

FIG. 2 illustrates an embodiment of the implantable medical device of FIG. 1 showing locations of its components relative to adjacent main anatomical structures of a patient's body after implantation.

FIG. 3 illustrates an embodiment of the implantable medical device of FIG. 1 showing a pump and a cleaning port of the device placed over abdominal muscles of the patient's body.

FIGS. 4-11 illustrate an embodiment of operations of the implantable medical device of FIG. 1.

FIG. 4 shows entrance of urine into tubes connected to the patient's ureters.

FIG. 5 shows the urine reaches a collection container placed in the patient's abdominal cavity.

FIG. 6 shows a pump exerting a negative pressure to cause the urine to flow into the pump through a connection tube between the collection container and the pump.

FIG. 7 shows the pump being filled up with the urine.

FIG. 8 shows effect of external pressure applied on the pump to allow the urine to exit the pump and flow through a tube that connects the pump to the urethra of the patient for final expulsion of the urine.

FIG. 9 shows a cleaning port filled with a liquid cleaning agent.

FIG. 10 shows the liquid cleaning agent entering through a tube connecting the cleaning port to the collection container.

FIG. 11 shows the entire implantable medical device being washed using the liquid cleaning agent.

FIGS. 12-20 illustrate embodiments of the implantable medical device of FIG. 1 with various additional features and design and application techniques.

FIG. 12 illustrates an embodiment of an implantable medical device with anti-reflux valves to prevent urine from flowing back to kidneys and portions of an environment in which the implantable medical device is used as an artificial bladder.

FIG. 13 illustrates an embodiment of an anti-reflux valve, such as the anti-reflux valve of FIG. 12.

FIG. 14 illustrates an embodiment of a high-pressure gradient valve, such as a valve used in the implantable medical device of FIG. 12 for preventing accidental urine dripping.

FIG. 15 illustrates an embodiment of an end portion of a ureteral tube of an implantable medical device, such as the implantable medical device of FIG. 12, to be inserted into a ureter.

FIG. 16 illustrates an embodiment of a secured connection between the ureteral tube and ureter of FIG. 15.

FIG. 17 illustrates an embodiment of a method for expanding a ureter in preparation for connecting the ureteral tube of FIG. 15 to the ureter.

FIG. 18 illustrates an embodiment of a method for preparing a purse-string suture for securing the connection between the ureteral tube of FIG. 15 and the ureter.

FIG. 19 illustrates an embodiment of an end portion of a urethral tube of an implantable medical device, such as the implantable medical device of FIG. 12, to be inserted into a urethra.

FIG. 20 illustrates an embodiment of a secured connection between the urethral tube and urethra of FIG. 19.

FIG. 21 illustrates an embodiment of a method for replacing functions of a urinary bladder using an implantable medical device.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical, and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, an implantable medical device that can be used as an artificial bladder for placement in the body of a patient to collect and discharge biological urinary fluids. In light of what is discussed in the BACKGROUND section, use of artificial bladder replacement prostheses made with suitable synthetic materials has undoubted advantages. However, despite research efforts in this field, there is still a need for such a device with satisfactory performance in the medium and long terms.

This is mainly due to the fact that despite having highly bio-compatible materials including medical grade silicone, challenges remain in developing an artificial bladder that avoids reflux, contains urine, and then expels the urine through the urethra at times controlled by the patient. Another challenge is the potential formation of a fungal-based microfilm on the interior walls of the artificial bladder that promotes infection and stone formation.

The urine, which is in continuous contact with the material inside the artificial bladder during its use, is a liquid that tends to be acidic, which exposes all kinds of material, to a greater or lesser extent, to the risk of fouling. Use of bioengineered tissues to reconstruction of the bladder have been investigated. Studies used regenerated tissues consisting of autologous patient cells that were cultured in vitro and then seeded in media, such as those made with collagen and polyglycolic acid, to construct organs for implantation. Despite the encouraging results from early experimental use of such regenerated tissues, their extensive clinical use still requires substantial efforts to gain knowledge and make advances in areas such as optimization of the regenerative materials to be used, cellular phenotypes to be selected, fixation and integration of implants in the human body, and so on.

On the other hand, many elastomeric materials have been proposed which exhibit mechanical properties, both static and dynamic, that are suitable for use as a substitute for natural bladder tissue. The present subject matter uses biocompatible materials that are known for reducing rate of potential infection and potential formation of stones and are known for suitability for long-term use with corrosion-resistance to urinary fluids. However, identifying a suitable material is just one of the fundamental goals for developing the implantable medical device for use as the artificial bladder.

In view of the efforts made over the past several decades in the fields of tissue engineering, materials science, and regenerative medicine, it is recognized by the inventor that a new approach to the structural and functional design of the implantable medical device for use as the artificial bladder is critical to restoration of the functions of the genitourinary system for improving the quality of life of the patients after removal of their natural bladders. Goals for this new approach include meeting requirements or desires of this technical field including, but not limited to ease of implantation in the patient's body, operation based on controllable urine emptying, adequate stability and biocompatibility that allows for long-term use after implantation, protection of normal kidney function, and cost effectiveness.

Pathologies related to the bladder showing its loss of control or inability to contract, such as in the case of the hypotonic bladder or the neurogenic bladder provoke considerations of replacing such lost or impaired functions using mechanical means in the implantable medical device. The inventor also discovered that a function of the natural bladder has been neglected though it seemed obvious, which is the ability of the bladder to notify the brain of the need to urinate based on the volume of the urine accumulated in the bladder. The human brain receives this notification not only through the internal nerves between organs, but also through indoctrinated images. While this function is missing from various existing artificial bladders, the present subject matter incorporates it into the implantable medical device for use as the artificial bladder.

The present subject matter provides an implantable medical device that can be used for replacing a natural organ that has been removed, for example due to cancer, severe trauma, or substantial failure of its function. When being used as an artificial bladder, the present implantable medical device can be made of a biocompatible material that is highly corrosion-resistant to urinary fluids and can have a shape and volume similar to those of the natural bladder.

In various embodiments, the present implantable medical device when used to replace the natural bladder of a patient can have a performance comparable to that of the natural bladder in terms of urine collection, reflux prevention, and expulsion of the collected urine through the patient's urethra. The present implantable medical device can be constructed for long-term corrosion resistance and stability in its intended environment inside the patient's body. Unlike other devices designed for the same or similar functions, the present implantable medical device mobilizes the urine out of the abdomen by means of a suction and expulsion pump operable by light pressure from the patient when desired. The pump is placed subcutaneously to allow the patient to apply pressure to draw the urine collected in a collection container placed in the abdominal cavity and move the urine from the pump back into the abdominal cavity to reach the urethra, which is a final conduit of the natural urination. Thus, the patient is allowed to control when they should and want to urinate and to urinate through the natural urethra, thereby maintaining a good quality of life. In various embodiments, the present implantable medical device is made radiation compatible because, for example, some of the patients may be subjected to radiation therapies. In various embodiments, the present implantable medical device can provide an option of being washed internally and frequently to minimize the risks of microfilm and/or stone formation.

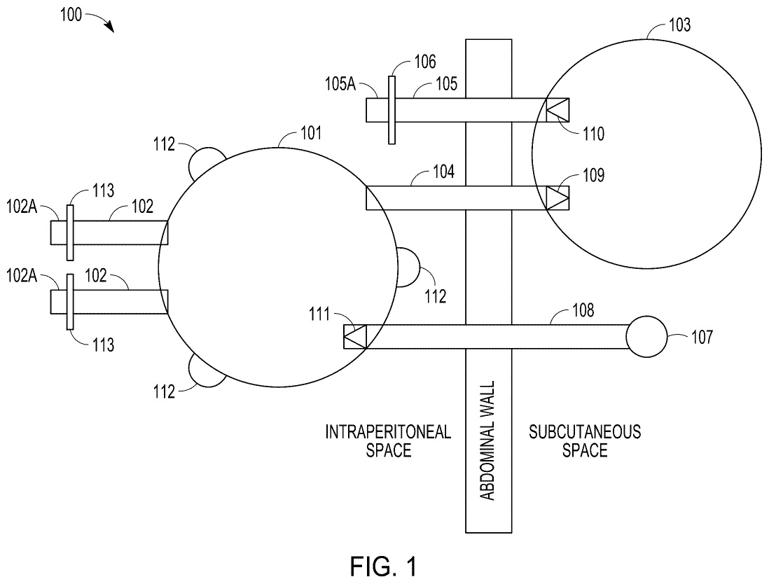

FIG. 1 illustrates an embodiment of an implantable medical device 100 and portions of an environment in which device 100 is used as an artificial bladder. Implantable medical device 100 can be used to restore functions of the natural bladder of a patient after the natural bladder is removed. To restore the functions of the natural bladder, implantable medical device 100 includes a collection container 101 for collecting urine, flaps 112 (optional, for affixing collection container 101 to tissue), two ureteral tubes 102 for connecting the ureters to collection container 101 (each including a portion 102A to be inserted into one of the ureters), two ureteral rings 113 (optionally) to secure the connections each between one of ureteral tubes 102 and one of the ureters, a pump 103, a connection tube 104 coupled with a unidirectional connection valve (also referred to as connection check valve) 109 for connecting collection container 101 to pump 103, a urethral tube 105 coupled with a unidirectional urethral valve (also referred to as urethral check valve) 110 for connecting pump 103 to the urethra (including a portion 105A to be inserted into the urethra), and a urethral ring 106 (optionally) to secure the connection between urethral tube 105 and the urethra. To allow for internal cleaning, implantable medical device 100 can optionally further include a cleaning port 107 and a cleaning tube 108 coupled with a unidirectional cleaning valve (also referred to as cleaning check valve) 111 for connecting port 107 to collection container 101. Implantable medical device 100 can be made entirely, or mostly, of medical grade silicone. This material is compatible with radiotherapy and shows excellent stability in its relationship with human tissues in the long term.

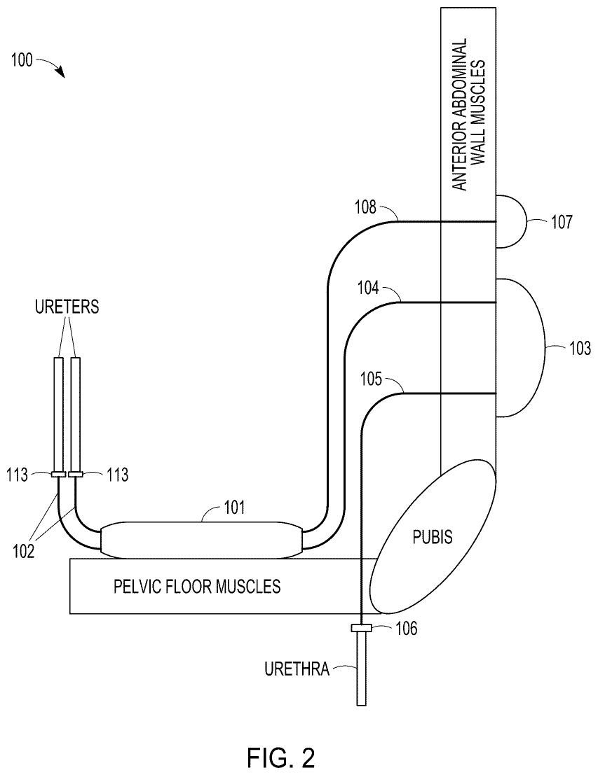

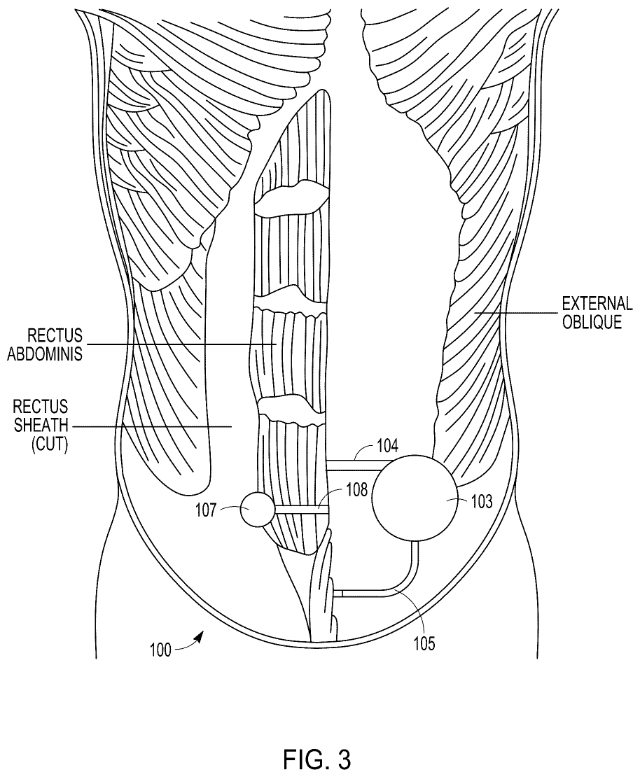

FIG. 2 illustrates an embodiment of implantable medical device 100 showing locations of its components relative to main adjacent anatomical structures of the of the patient's body after implantation of implantable medical device 100 into the patient. The anatomical structures shown in FIG. 2 include representations of muscles of the pelvic floor, the pubis (bone), and muscles of the anterior abdominal wall. FIG. 3 illustrates an embodiment of implantable medical device 100 showing its components that are subcutaneously placed between the abdominal wall and the skin, external to the abdominal cavity, after the implantation of implantable medical device 100 into the patient. FIG. 3 shows pump 103 and port 107, with portions of tubes 104, 105, and 108 placed on a portion of the rectus sheath (illustrated with a cutaway portion to show the underneath rectus abdominis) and over portions of the rectus abdominis (and possibly portions of the external oblique). A unique and fundamental characteristic of implantable medical device 100 is that parts of the device are implanted inside the abdominal cavity while other parts of the device are implanted outside the abdominal cavity in the subcutaneous space over the abdominal wall muscles, which allows for patient control of the urination process.

Collection container 101 is a bag having a flexible, elastic hollow body for use as a passive urine container that can have a volume capacity between 100 ml and 350 ml, with approximately 250 ml being a specific example. The volume capacity can be custom selected for the patient. Collection container 101 can have shape and size similar to those of the natural bladder (e.g., a spherical shape or a shape more closely resembling that of the natural bladder) and can be placed within the abdominal cavity, in the space previously occupied by the natural bladder (which has been removed). Thus, collection container 101 can be placed inside the patient's abdominal cavity to resemble the natural anatomical position and structure of the bladder. Affixation features can be incorporated onto collection container 101 to stabilize its position in the abdominal cavity after the implantation of device 100. In one embodiment, as illustrated in FIG. 1, collection container 101 includes flaps 112 that can be sutured to deep tissues (e.g., muscles of the pelvic floor) to minimize displacement of collection container 101 in the abdominal cavity.

Ureteral tubes 102 are flexible conduits each have one end coupled to collection container 101 and the other end to be connected to one of the patient's ureters, to provide for fluid communication from the ureters to collection container 101. Collection container 101 receives the urine from the patient's kidneys through the ureters and ureteral tubes 102. In this manner, collection container 101 functions as a passive urine container. Ureteral rings 113 can each include one or more rings optionally coupled to the respective ureteral tube 102 for providing a secure connection between that tube and the respective ureter. For example, when needed, ureteral rings 113 can each be sutured to the respective ureter after portion 102A of the respective ureteral tube 102 has been inserted into that ureter.

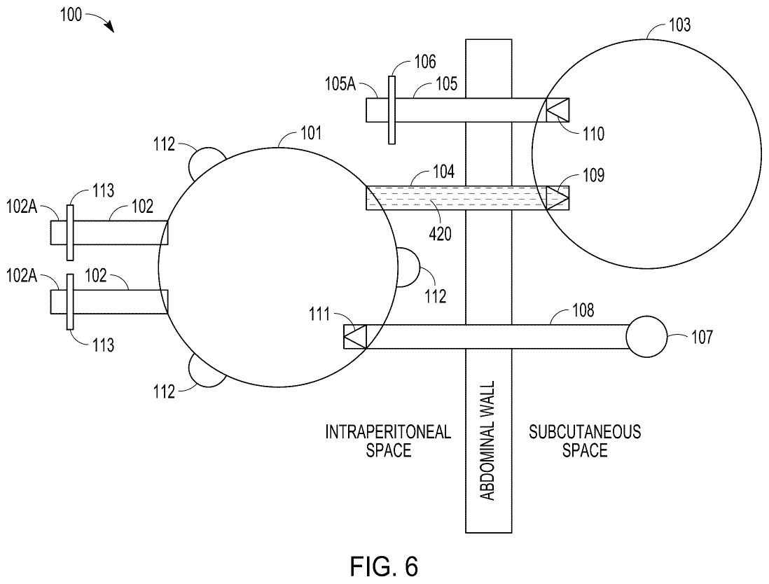

Pump 103 includes an elastic container that can be pressed to function as a pump. This container of pump 103 can have a volume capacity between 50 ml and 350 ml, with approximately 250 ml being a specific example. This volume capacity can be custom selected for the patient when needed. For example, while 250 ml is considered to be sufficient given average normal production of a urination, a larger volume (e.g., 300 ml) can be used if the patient anticipates higher-than-average urine production rate and/or longer urination periods for any reason. The hollow container can have a wall that is sufficiently thick to maintain a shape (e.g., a hemispherical shape) after device 100 is implanted. The thickness of the wall can be between 3 mm to 6 mm, with 4 mm being a specific example. Pump 103 is to be subcutaneously placed outside of the abdominal cavity and can be affixed (e.g., sutured) to the fascia of the abdominal muscles. In various embodiments, the wall of a base portion of pump 103 (the flat portion to be attached to the fascia of the abdominal muscles) is substantially (e.g., over 50%, 75%, 100%, 125%, 150%, 175%, or 200%) thicker than the wall of a hemispherical top portion of pump 103. For example, the wall of the flat base portion of pump 103 can be up to twice as thick as the wall of the hemispheric top portion of pump 103. When the wall of the hemispherical top portion is 4 mm thick, the wall of the flat base portion can be up to 8 mm thick. The thicker flat base portion of pump 103 facilitates exertion of a negative pressure for the pumping function as further discussed below (with reference to FIG. 6).

Connection tube 104 is a flexible conduit connecting pump 103 to collection container 101 to provide fluid communication from collection container 101 pump 103. Unidirectional connection valve 109 is coupled to connection tube 104 to allow the urine to flow through connection tube 104 in the direction from collection container 101 to pump 103 only.

Urethral tube 105 is a flexible conduit having one end connected to pump 103 and the other end to be connected to the patient's urethra to provide for fluid communication from pump 103 to the urethra. Unidirectional urethral valve 110 is coupled to urethral tube 105 to allow the urine to flow through urethral tube 105 in the direction from pump 103 to the urethra only. Urethral ring 106 can include one or more rings optionally coupled to urethral tube 105 for providing a secure connection between urethral tube 105 and the urethra. For example, when needed, urethral ring 106 can be sutured to the urethra neck after portion 105A of urethral tube 105 has been inserted into the urethra.

Implantable medical device 100 can be implanted into the patient with pump 103 producing a spontaneous vacuum sucking the urine from collection container 101 through connection tube 104 and connection valve 109. This negative pressure exists until the container of pump 103 reaches its volume capacity and opens unidirectional connection valve 109 such that the urine can only flow from collection container 101 (functioning as a passive container) to pump 103 (functioning as an active container). The patient can perceive that the urine contained in pump 103 approaches its volume capacity, which is the mechanism of implantable medical device 100 for notifying the patient to empty the urine. For example, the patient can see and/or fell pump 103 as a bump that looks like a small breast implant when it is full and a small crater when it is empty. Once in the bathroom, the patient can gently press the skin over pump 103 with a hand. This pressure causes connection valve 109 to close and urethral valve 110 to open, thereby causing the urine in pump 103 to be expelled from the patient through urethral tube 105 and the urethra.

Once the patient finishes urinating, pump 103 can return spontaneously to its starting empty state by producing a slight and continuous vacuum inside it, which closes unidirectional urethral valve 110 and opens connection valve 109 when urine again accumulates in collection container 101. All the urine that reaches the intra-abdominally positioned collection container 101 will be constantly sucked into the extra-abdominally positioned pump 103, such that the patient can participate in the urination process in a manner similar to that with the natural bladder. Valves 109 and 110 can each be positioned in an un-collapsible area (e.g., within pump 103) to avoid interference during the suction and expulsion processes of pump 103.

In various embodiments, implantable medical device 100 optionally includes an internal cleaning mechanism that include cleaning port 107, cleaning tube 108, and unidirectional cleaning valve 111. Cleaning port 107 includes an elastic container that allows for injecting a liquid cleaning agent into the urination circuit of implantable medical device 100 through cleaning tube 108 and cleaning valve 111. Cleaning tube 108 is a flexible conduit connecting cleaning port 107 to collection container 101, to provide for fluid communication from cleaning port 107 to collection container 101. Unidirectional cleaning valve 111 is coupled to cleaning tube 108 to allow the liquid cleaning agent to flow through cleaning tube 108 in the direction from cleaning port 107 to collection container 101 only. Cleaning valve 111 can be positioned in an un-collapsible area (e.g., within collection container 101).

Cleaning port 107 is to be placed subcutaneously over the abdominal wall such that it is visible and palpable through the abdominal skin after implantable medical device 100 is implanted. A cleaning procedure can be performed percutaneously by using a thin surgical stainless steel lead (e.g., a hollow needle) to inject the liquid cleaning agent into cleaning port 107. The wall of cleaning port 107 can be self-sealing after being pierced for the injection of the liquid cleaning agent. The liquid cleaning agent can include a substance that changes the pH value of the content inside implantable medical device 100 to clean and disinfect the interior surfaces of implantable medical device 100 against potential infection or contamination caused by the residual urinary liquid. An example of the liquid cleaning agent includes a non-irritant mixture of saline solution with sodium hypochlorite and large spectrum antibiotics. Each cleaning process can start with injecting about 100 ml of his solution into cleaning port 107. Once the liquid cleaning agent is injected (and mixed with the urine in collection container 101), the patient can perform the same procedure as in the urination process to complete the cleaning process with the liquid cleaning agent expelled through the urethra. The liquid cleaning agent cleans the interior wall surfaces of all the components of implantable medical device 100 during the cleaning process.

In various embodiments, unidirectional valves 109, 110, and 111 are each used to prevent the urine from flowing in the wrong direction and/or into a wrong portion of implantable medical device 100 or the patient (e.g., the kidneys). In various embodiments, while valves 109, 110, and 111 are each coupled to one end of its respective tube, they can each be coupled with to the respective tube in any location for providing the tube with the intended unidirectionality.

Implantable medical device 100 can be seen as an artificial passive bladder connected to a manually operated active compression pump to collect the patient's urine and allows the collected urine to be controllably expelled through the urethra. A mechanism for cleaning can be included in implantable medical device 100 when desired, for an easy and convenient way of allowing for periodic maintenance ensuring proper functioning of implantable medical device 100 during its intended long-term use. In one embodiment, implantable medical device 100 is constructed and provided for surgical implantation as a single unit. In other embodiments, implantable medical device 100 is constructed as multiple components that can be assembled prior to and/or during surgical implantation.

In various embodiments, implantable medical device 100 is made of 100% biocompatible material. Biocompatibility of a material used to construct a device intended to be implanted in a biological system includes acceptable types and degrees of interactions between the material and the host biological system. This includes an appropriate response of the host biological system to the material for a given application of the device, including not interfering or detrimentally interacting with the physiological activities of the host biological system. Interactions between the external surface of implantable medical device 100 and the surrounding tissue result in minimal inflammatory response.

In various embodiments, the biocompatible material used for constructing implantable medical device 100 is also reliable for long-term implantation. Such a material is biostable. Once implantable medical device 100 is implanted, the material does not undergo substantial chemical and/or chemical transformation over time. While “long-term” can include any length of time over 30 days, in various embodiments implantable medical device 100 is intended for use in the patient for at least 10 years. In various embodiments, a preventive maintenance that can include replacement of implantable medical device 100 can be performed periodically following the implantation, such as every 10 years, and/or as needed (e.g., indication of device dysfunction or side effect observed).

In various embodiments, the biocompatible material used for constructing implantable medical device 100 has mechanical properties comparable to these of the natural organ it is intended to replace. For use as the artificial bladder, the material has mechanical properties suitable for supporting the functionality of implantable medical device 100 including accumulation and discharge of urinary body fluids.

In various embodiments, implantable medical device 100 is made of materials having the biocompatibility, reliability, and mechanical properties as discussed above. One example of such material is medical grade silicone. In one embodiment, implantable medical device 100 is entirely, or mostly (e.g., entirely with exception of one or more relatively small components), made of medical grade silicone.

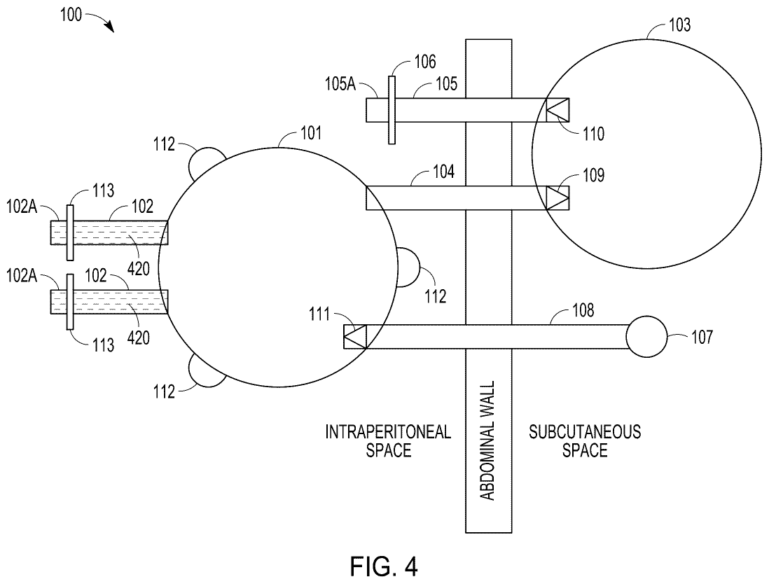

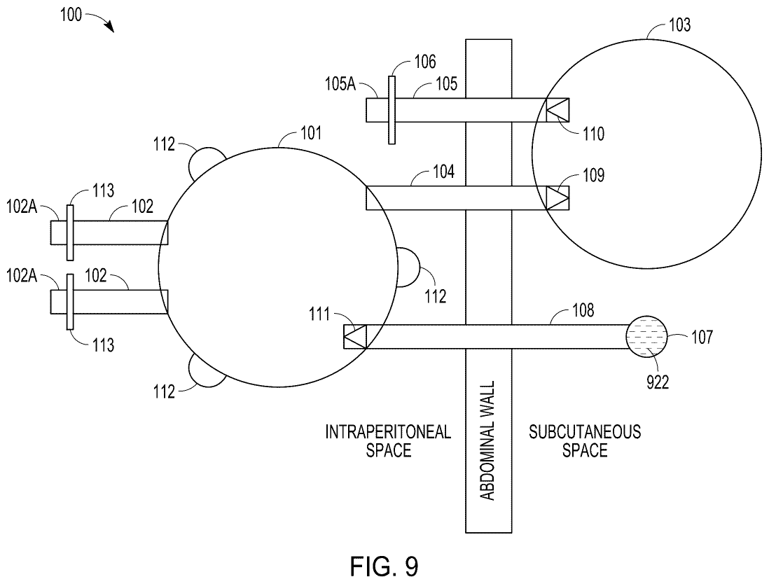

FIGS. 4-11 illustrate an embodiment of operations of implantable medical device 100 (including the optional cleaning mechanism), showing a urination cycle (FIGS. 4-8) and a cleaning cycle (FIGS. 9-11). The shaded areas in FIGS. 4-8 indicate where the urine (420) reaches. The shaded areas in FIGS. 9-11 indicate where the liquid cleaning agent (922) reaches.

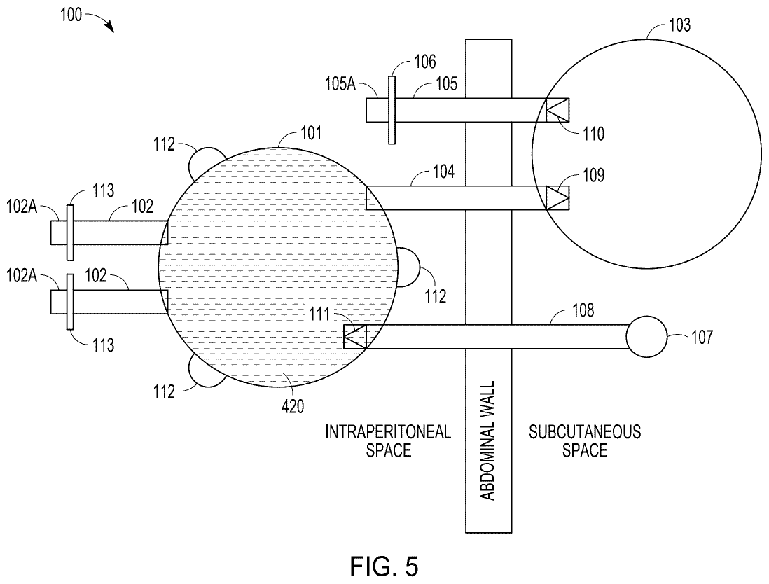

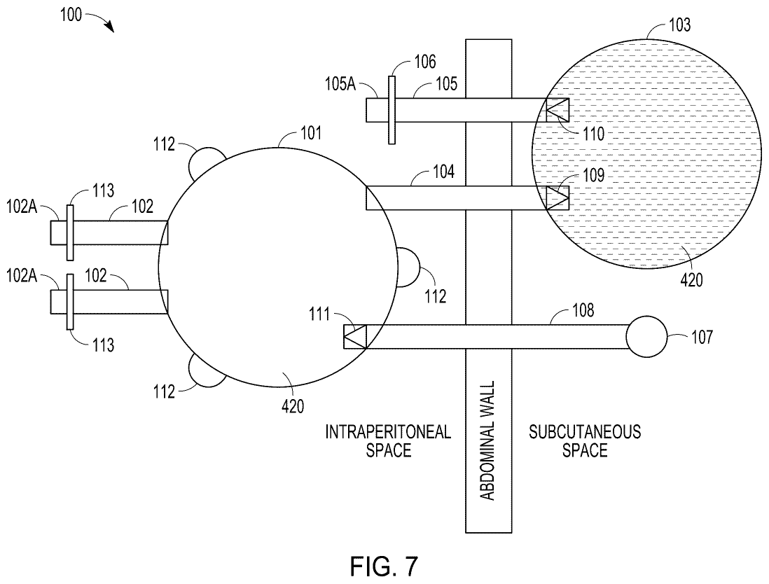

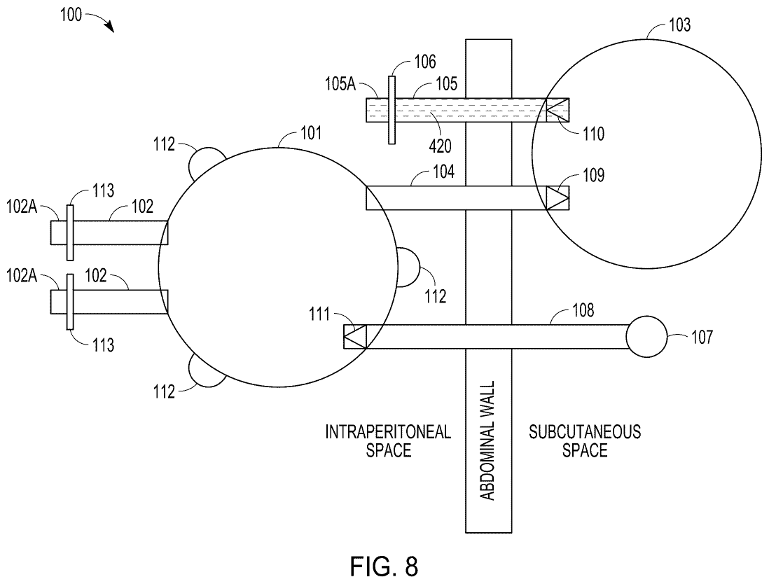

FIG. 4 shows entrance of urine 420 into ureteral tubes 102 connected to the patient's ureters. FIG. 5 shows urine 420 reaches collection container 101, which is placed in the intraperitoneal space of the patient's abdominal cavity. FIG. 6 shows pump 103 exerting a negative pressure to cause urine 420 to flow into pump 103, which is placed in the subcutaneous space outside of the abdominal cavity, through connection tube 104 and unidirectional connection valve 109. Connection valve 109 prevents urine 420 from returning to collection container 101 after it reaches pump 103. FIG. 7 shows pump 130 being filled with urine 420. When pump 130 is substantially (e.g., over 60%, 70%, 80%, or 90%) filled or fully filled, the patient can see and/or feel the need for urination. FIG. 8 shows the effect of external pressure applied on pump 130 to allow urine 420 to exit pump 130 and flow through urethral tube 105 and unidirectional urethral valve 110 for final expulsion of urine 420 through the patient's urethra. Unidirectional urethral valve 110 is opened under the pressure, such that urine flows from pump 103 to urethral tube 105 only when pump 103 is under the external pressure, and prevents urine 420 from returning to pump 130 after it enters urethral tube 105.

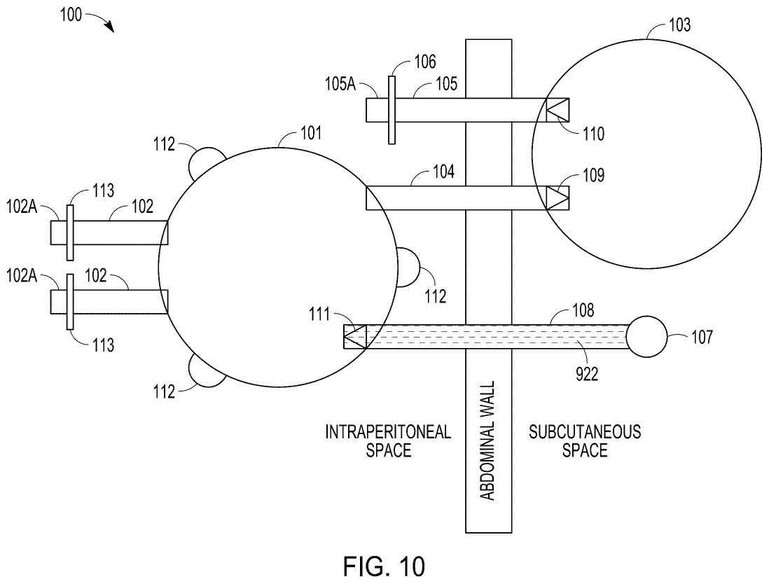

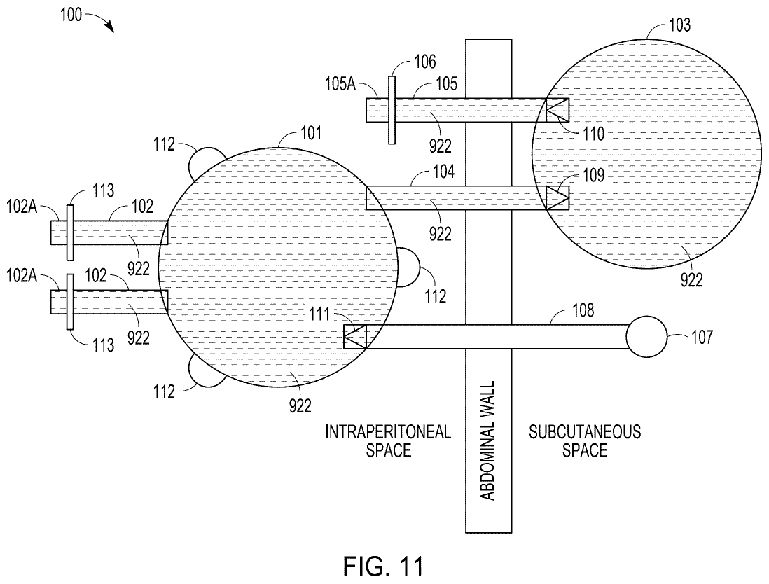

FIG. 9 shows cleaning port 107 being filled with liquid cleaning agent 922. The liquid cleaning agent can be injected into cleaning port 107 using a sharp hollow needle or lead that can pierce the wall of cleaning port 107, and the wall can be made self-sealing after being pierced. FIG. 10 shows liquid cleaning agent 922 enters collection container 101 through cleaning tube 108 and unidirectional cleaning valve 111. Cleaning valve 111 prevents liquid cleaning agent 922 from returning to cleaning tube 108 after it reaches collection container 101. FIG. 11 shows the entire interior space of the urination circuit (show by the shaded areas) of implantable medical device 100 being washed using liquid wash agent 922. Once liquid cleaning agent 922 enters collection container 101, the process of washing and expulsion of liquid cleaning agent 922 resembles that of the urination cycle as illustrated in FIGS. 4-8.

The present subject matter is unique in terms of active support from the patient to expel the urine through the patient's natural, intact urethra. Once pump 103 is full or nearly full, the patient can see and/or feel it through the skin in the abdominal region. A gentle pressure with the fingers with the support from the abdominal muscle wall causes the expulsion of the urine.

The present subject matter provides a completely artificial and long-term stable bladder that does not require removal of part of the patient's intestine or other tissues of the patient, thus eliminating the associated risks, complications, and loss of performance over time. When compared to the use of an external bag attached to the abdomen, the present subject matter offers significant advantages in the patient's quality of life by, for example, solving problems associated with the external bag when participating in social activities.

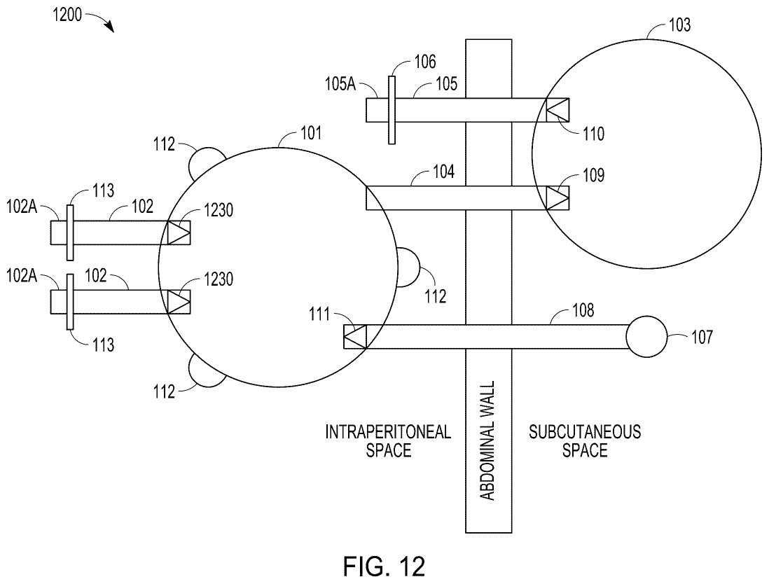

Various structural features can be added, and various design and application techniques can be applied to implantable medical device 100 to enhance its functionality and/or its interface with the patient's natural anatomy. Examples of such features and techniques are discussed below with reference to FIGS. 12-20. In various embodiments, any one or any combination of one or more of these examples can be applied to implantable medical device 100.

### Anti-Reflux Valves in Collection Container

FIG. 12 illustrates an embodiment of an implantable medical device 1200 and portions of an environment in which implantable medical device 1200 is used as an artificial bladder. Implantable medical device 1200 represents an example of implantable medical device 100 in which anti-reflux valves 1230 are added to prevent urine from flowing back to the kidneys. In a normal body, a natural unidirectional valve system at the junction of the ureters and the bladder prevents urine from backing up into the kidneys, for example when the patient is in the supine position. In implantable medical device 1200, anti-reflux valves 1230 can provide for this function. Anti-reflux valves 1230 are each coupled to one of ureteral tubes 102 and positioned in collection container 101 to prevent urine from backing up into the kidneys once it is in collection container 101. In various embodiments, anti-reflux valve 1230 can be a passive valve, such as a duckbill valve or a capillary valve, that allows fluid to flow in one direction. Pressure inside the valve can prevent the fluid from flowing in the opposite direction.

FIG. 13 illustrates an embodiment of an anti-reflux valve 1330, which represents an example of anti-reflux valve 1230. Anti-reflux valve 1330 is a duckbill valve having a proximal end portion 1329, a distal end portion 1331, and a tapered transitional portion between proximal end portion 1329 and distal end portion 1331. Proximal end portion 1329 allows anti-reflux valve 1330 to be securely connected to the end portion of one of ureteral tubes 102 (e.g., by elastic force and/or cement). Distal end portion 1329 is positioned in collection container 101 when anti-reflux valve 1330 is connected to the one of ureteral tubes 102. Urine can flow through anti-reflux valve 1330 from proximal end portion 1329 to distal end portion 1331, while pressure inside the valve can prevent the urine from flowing in the opposite direction. When used as each of anti-reflux valves 1230, the duckbill valve allows urine to flow effortlessly from the kidneys to collection container 101 but prevents urine in collection container 101 from returning to the kidneys through each of the ureters. Anti-reflux valve 1330 can be made of silicone, which can be the same material used to construct most (e.g., over 90%) or substantially the entirety (e.g., over 95%, 96%, 97%, 98%, or 99%) of implantable medical device 1200 and can allow anti-reflux valve 1330 to be securely connected to the end portion of ureteral tube 102 by elastic force from proximal end portion 1329.

### Resilience of Collection Container

To function properly, collection container 101 cannot collapse when intra-abdominal pressure increases as anticipated in the patient's daily activities. Like the natural bladder, collection container 101 should maintain a level of pressure that causes urine to flow from the kidneys to the collection container without dependent on passive flow of the urine. In the natural urinary system, wave-like muscular contractions known as ureteral peristalsis propel urine from the kidneys to the bladder through the ureters, overcoming the resistance of the natural anti-reflux valve of the bladder.

Collection container 101 can be configured to have a degree of resilience suitable for maintaining an internal pressure to actively draw urine from the kidneys to collection container 101 through the ureters, ureteral tubes 102, and anti-reflux valves 1230. The internal pressure is the pressure in the internal cavity of collection container 101. When the internal pressure is lower than the pressure inside the kidneys, a suction force is created to overcome the resistance of anti-reflux valves 1230 to draw urine from the kidneys through the ureters, ureteral tubes 102, and anti-reflux valves 1230. Given the material (e.g., medical grade silicone) used to construct collection container 101, a required or desirable degree of resilience can be provided by determining a thickness of the wall of collection container 101.

Intra-abdominal pressure may compress collection container 101, for example when the patient is in a lateral recumbent position. When that pressure is removed, collection container 101 can return to its original shape due to the resilience provided at least in part by the thickness of its wall. The resilience also creates a low pressure in the internal cavity of collection container 101 to result in a suction force that overcome the resistance of anti-reflux valves 1230 to draw urine from the kidneys through the ureters and ureteral tubes 102. Unidirectional connection valve 109 prevents urine from flowing back to collection container 101 from pump 103 while contributing to the generation of the suction force.

The wall thickness of collection container 101 can be determined analytically and/or empirically. For example, computerized analysis can be employed to determine the required degree of resilience and to translate the resilience into the wall thickness based on known characteristics of its material (e.g., medical grade silicone) and computational models of collection container 101 and various pressures anticipated. Empirical determination can include testing instances of collection container 101 having different wall thicknesses subjected to various types and levels of pressures simulating what collection container 101 may subject to inside the patient.

In one embodiment, collection container 101 is constructed (of medical-grade silicone for example) to have a wall thickness and elastomeric memory determined and calibrated to exert a continuous and spontaneous negative pressure (i.e., a vacuum-like internal pressure that creates a suction force). The degree of resilience of collection container 101 can maintain the negative pressure of approximately (e.g., within 5%, 10%, 15%, 20%, or 25% of) 15 cm of water (cmH₂O) during periods of patient inactivity or sleep. Such a negative pressure allows urine to be actively drawn from the renal pelvises of the kidneys to collection container 101 (through the ureters, ureteral tubes 102, and anti-reflux valves 1230), thereby preventing urinary stasis in the kidneys and overcoming the resistance of anti-reflux valves 1230, regardless of the patient's posture.

### High-Pressure Gradient Valve

Urine is discharged from the body in response to a pressure applied on pump 103 exceeding a threshold pressure level, which can be set for preventing accidental discharge of the urine. Unidirectional urethral valve 110 can be a high-pressure gradient valve having an opening pressure determined for allowing flow of urine from pump 103 to urethral tube 105 when pump 103 is intentionally pushed for urination while preventing the urine from being accidentally released from pump 103 to urethral tube 105. The high-pressure gradient valve can be opened to allow flow of the urine from pump 103 to urethral tube 105 only when the pressure inside pump 103 reaches a level corresponding to the opening pressure of the urinary sphincter in a normal natural urinary system (e.g., about 60 cmH₂O). This prevents accidental pressures (i.e., pressures not resulting from an intention to urinate, such as pressuring resulting from the patient is in a prone position) from opening unidirectional urethral valve 110 to result in unintended urination. Other unidirectional valves in implantable medical device 100 or 1200 can have lower opening pressures, such as about 20 cmH₂O.

FIG. 14 illustrates an embodiment of a high-pressure gradient valve 1410 for preventing accidental urine dripping. High-pressure gradient valve 1410 can be an example of unidirectional urethral valve 110 and can include a valve body 1432 (e.g., made of plastic), a ring 1433 to provide for a secure connection to urethral tube 105, and a spring 1434 to provide the opening pressure.

### Secure Ureteral Connections

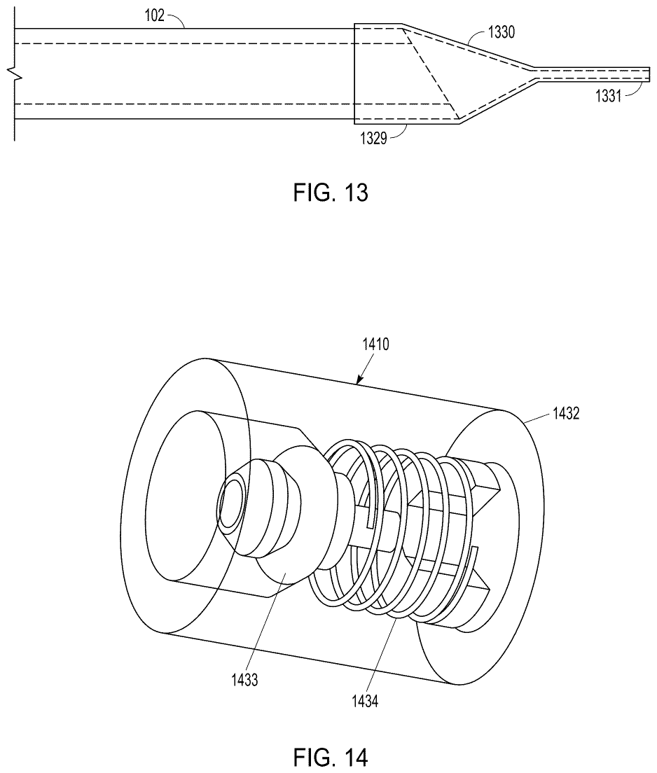

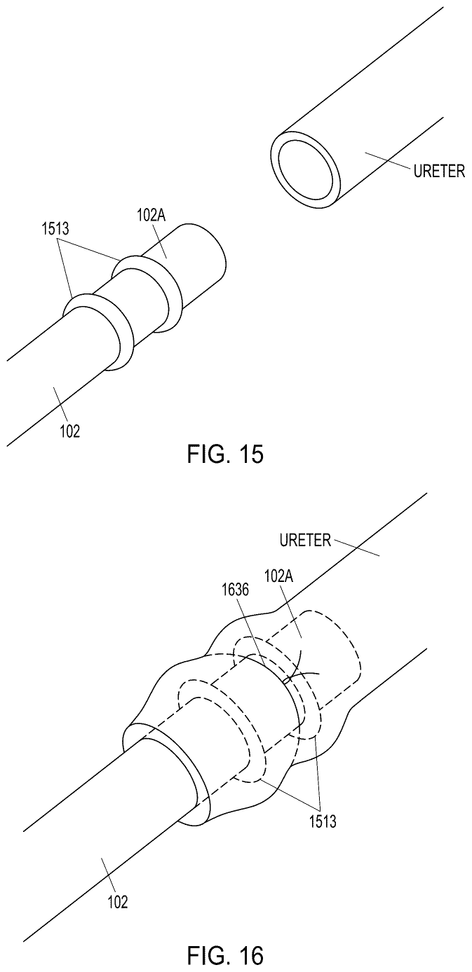

FIG. 15 illustrates an embodiment of an end portion of a ureteral tube of an implantable medical device, such as end portion 102A of ureteral tube 102 of implantable medical device 100 or 1200, that is to be inserted into a ureter. Implantable medical device 100 or 1200 includes two ureteral tubes 102 that can be connected to respective ureters of the patient. Two ureteral rings 1513 can be attached to each of ureteral tubes 102 at end portion 102A to secure a connection between that ureteral tube 102 and one of the ureters. Ureteral rings 1513 can be an example of ureteral rings 113 and can be made of silicone.

FIG. 16 illustrates an embodiment of the secured connection between ureteral tube 102 and the ureter. Each of ureteral tubes 102 can be secured to a ureter using a suture 1636 passing through the ureter wall between ureteral rings 1513. This technique is provided to avoid leakage of urine from the connections between ureteral tubes 102 and the ureters without injuring the ureteral microvasculature. Suture 1636 can be made of non-absorbable material. Ureteral rings 1513 are separated by a distance allowing each ureteral tube 102 to be secured to the respective ureter using suture 1636.

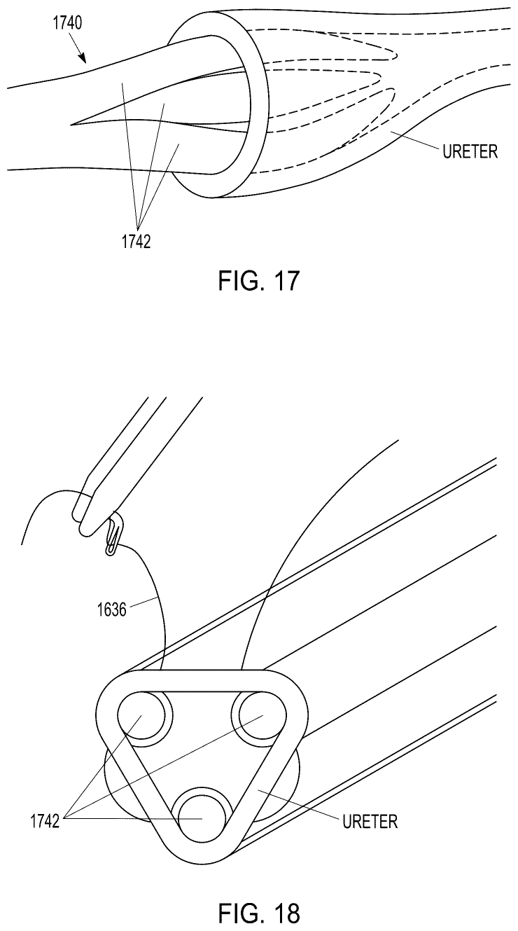

FIG. 17 illustrates an embodiment of a method for expanding a ureter in preparation for connecting one of ureteral tubes 102 to the ureter. A surgical instrument 1740 is to keep the ureter expanded while a drawstring suture (1636) is passed to tie the ureter to respective ureteral tube 102. In the illustrated embodiment, surgical instrument 1740 is an expansion tool having three prongs 1742. The expansion tool can be rigid, such as being made of stainless steel. During the operation of replacing the patient's dysfunctional natural bladder with implantable medical device 100 or 1200, the ureters are each disconnected from the natural bladder to allow the natural bladder to be removed from the patient. Once the bladder is removed, the ureters become open-ended in the abdomen. The surgeon grasps the end portion of each ureter and inserts prongs 1742 of surgical instrument 1740 to expand the lumen of that ureter.

FIG. 18 illustrates an embodiment of a method for preparing a purse-string suture for securing the connection between the one of ureteral tubes 102 and the respective ureter. Prongs 1742 of surgical instrument 1740 are configured to dilate the lumen as well as the circumference of each of the ureters to allow suture 1636 to be pass the wall of that ureter into the lumen and back to the outside of the ureter. This maneuver is repeated to pass suture 1636 under each of prongs 1742 of surgical instrument 1740, as a form of a purse-string suture (i.e., a surgical technique used to close circular wound by creating a running stitch around the perimeter of the wound and cinch like a drawstring to close the wound). Once completed, surgical instrument 1740 is removed, and each of ureteral tubes 102 is inserted into the lumen of the respective ureter. The surgeon then tightens and ties the suture, thereby securing each of ureteral tubes 102 to the respective ureter to prevent ureteral tubes 102 from displacement relative to the respective ureters to which they are connected. Subsequently, the natural healing process in the patient includes development of scar and neovasculature from the ureteral epithelium to seal the connections between ureteral tubes 102 and the ureters to prevent urine from leaking through these connections.

### Secure Urethral Connection

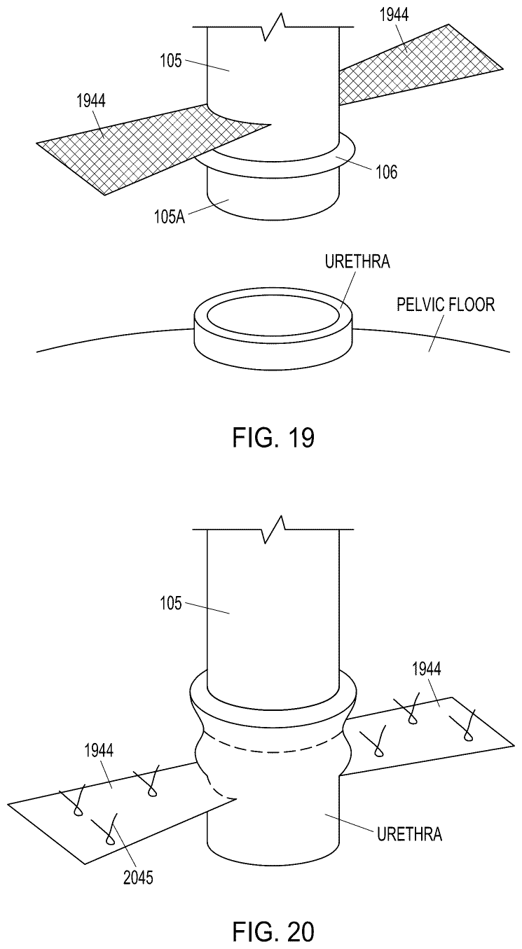

FIG. 19 illustrates an embodiment of an end portion of a urethral tube of an implantable medical device, such as end portion 105A of urethral tube 105 of implantable medical device 100 or 1200, that is to be inserted into a urethra. During the operation of replacing the patient's dysfunctional natural bladder with implantable medical device 100 or 1200, the urethra is disconnected from the dysfunctional bladder to allow the bladder to be removed from the patient and becomes open-ended. Then, end portion 105A of urethral tube 105 is inserted into the urethra to form a connection between urethral tube 105 and the urethra. In the illustrated embodiment, extensions 1944 are affixed to urethral tube 105 to allow for fixation of urethral tube 105 by suturing extensions 1944 onto the pelvic floor of the patient and allowing for healing with neovasculature. The pelvic floor (also known as pelvic diaphragm) is an anatomic structure including muscles, ligaments, and connective tissues that form the base of the pelvis and support the bladder and controlling the release of urine. Fixation of urethral tube 105 to the pelvic floor can secure the connection between urethral tube 105 and urethra against movements of surrounding tissue such as during exercise or trauma. Extensions 1944 can be a mesh extension constructed of titanium or titanium alloy or one or more other suitable materials.

End portion 105A can be sutured directly to the urethra to form the connection between urethral tube 105 and the urethra. In various embodiments, urethral tube 105 can be sutured to the edge of the open end of the urethra and to the pelvic floor. An enhanced connection between urethral tube 105 and the urethra can be provided by inserting end portion 105A of urethral tube 105 into the urethra and suturing extensions 1944 to the muscles of the pelvic floor. Scar and neovasculature can develop around extensions 1944 to strengthen the connection and prevent urine from leading through the connection during exercise or trauma. Urethral ring 106 serves as a protective barrier, ensuring that the suture passes through it and does not injure the structure of urethral tube 105, thereby preventing possible leaks.

FIG. 20 illustrates an embodiment of a secured connection formed between urethral tube 105 and the urethra. Extensions 1944 can each be affixed onto the pelvic floor muscles using sutures 2045. In various embodiments, extensions 1944 can be connected to the urethra for additional support in securing the connection, along with their affixation onto the pelvic floor.

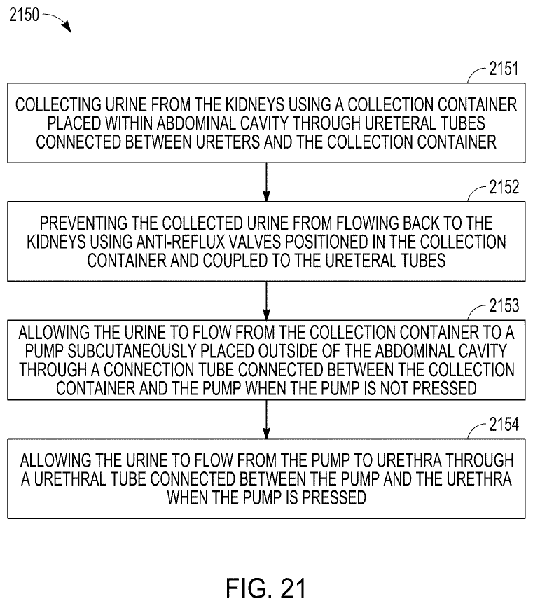

FIG. 21 illustrates an embodiment of a method 2150 for replacing functions of a urinary bladder using an implantable medical device. In one embodiment, method 2150 is performed using implantable medical device 1200. In one embodiment, the entire implantable medical device is constructed using medical grade silicone. In another embodiment, the implantable medical device is constructed using medical grade silicone except for one or more relatively small components.

At 2151, urine is collected from kidneys of a patient's body using a collection container placed within abdominal cavity of the body through first and second ureteral tubes connected between ureters of the body and the collection container. Connections each between a ureteral tube of the first and second ureteral tubes and a ureter of the ureters can be secured using two rings attached to an end portion of the ureteral tube inserted into the ureter and a suture passing through a wall of the ureter wall between the two ureteral rings and tied around the end portion of the ureteral tube.

At 2152, the collected urine is prevented from flowing back to the kidneys using first and second anti-reflux valves positioned in the collection container and coupled to the first and second ureteral tubes. An internal pressure in the collection container can be maintained using a degree of resilience of the collection container to actively draw the urine from the kidneys to the collection container through the ureters, the first and second ureteral tubes, and the first and second anti-reflux valves.

At 2153, the urine is allowed to flow from the collection container to a pump subcutaneously placed outside of the abdominal cavity through a connection tube connected between the collection container and the pump when the pump is not pressed. At 2154, the urine is allowed to flow from the pump to the urethra of the body through a urethral tube connected between the pump and the urethra when the pump is pressed. In various embodiments, the pump is “pressed” when the pressure applied on the pump exceeds a pressure level set using a high-pressure gradient valve positioned in the pump and coupled to the urethral tube. This ensures that each urination results from intentionally pressing the pump. Connection between the urethral tube and the urethra can be secured using a urethral ring attached to the urethral tube and/or using extensions affixed to the urethral tube and affixed to the pelvic floor of the body. The pump can be configured to allow the patient to perceive its fullness as an indication of need for urination.

In various embodiments, a method for cleaning the interior of the implantable device is additionally provided. A liquid cleaning agent can be received into the collection container for cleaning interior space of the implantable medical device. This can include receiving the liquid cleaning agent from a cleaning port subcutaneously placed outside of the abdominal cavity and connected to the collection container through a cleaning tube and receiving the liquid cleaning agent injected into the cleaning port using a hollow needle piercing the cleaning port. The liquid cleaning agent can include one or more substances for disinfection and prevention of bladder stone formation.

### Examples

Some non-limiting examples (Examples 1-40) of the present subject matter are provided as follows:
Example 1 is an implantable medical device for collecting and discharging urine from a body of a patient having kidneys, ureters, a urethra, an abdominal cavity, abdominal muscles having a fascia, and a pelvic floor. The implantable medical device can include: first and second ureteral tubes configured to be connected to the ureters to receive the urine from the kidneys through the ureters; a collection container coupled to the first and second ureteral tubes and configured to be placed inside the abdominal cavity and to receive the urine from the first and second ureteral tubes; first and second anti-reflux valves positioned in the collection container and respectively coupled to first and second ureteral tubes, the first and second anti-reflux valves configured to prevent urine from flowing from the collection container to the kidneys; a pump configured to be placed outside of the abdominal cavity; a connection tube connected between the collection container and the pump to provide for fluid communication from the collection container to the pump; a unidirectional connection valve coupled to the connection tube and configured to allow the urine to flow from the collection container to the pump; a urethral tube connected to the pump and configured to be connected to the urethra; and a unidirectional urethral valve coupled to the urethral tube and configured to allow the urine to flow from the pump to the urethra.
Example 2 is the implantable medical device according to Example 1, in which the first and second anti-reflux valves each include a duckbill valve.
Example 3 is the implantable medical device according to any of Examples 1 and 2, in which the collection container is configured to have a degree of resilience determined for maintaining an internal negative pressure to actively draw urine from the kidneys to the collection container through the ureters, the first and second ureteral tubes, and the first and second anti-reflux valves and to prevent urinary stasis in the kidneys.
Example 4 is the implantable medical device according to Example 3, in which the collection container has a wall, and a thickness of the wall is determined to provide the degree of resilience.
Example 5 is the implantable medical device according to any of Examples 1 to 4, in which the urine is discharged from the body in response to a pressure applied on the pump exceeding a threshold pressure level, and the unidirectional urethral valve includes a high-pressure gradient valve configured to have an opening pressure determined based on the threshold pressure level for allowing flow of the urine from the pump to the urethral tube.
Example 6 is the implantable medical device according to any of Examples 1 to 5, further including two ureteral rings attached to each ureteral tube of the first and second urethral tubes, the two ureteral rings configured and positioned to secure a connection between the each ureteral tube to a respective ureter of the ureters and to prevent the urine from leaking through that connection.
Example 7 is the implantable medical device according to Example 6, in which the two ureteral rings are separated by a distance allowing the each ureteral tube to be secured to the respective ureter using a suture passing through a wall of the ureter between the two ureteral rings.
Example 8 is the implantable medical device according to any of Examples 1 to 7, further including extensions affixed to the urethral tube, the extensions configured and positioned to be affixed to the pelvic floor after the urethral tube is inserted into the urethra.
Example 9 is the implantable medical device according to Example 8, in which the extensions include mesh extensions constructed of titanium or titanium alloy.
Example 10 is the implantable medical device according to any of Examples 1 to 9, further including a urethral ring attached to the urethral tube, the urethral ring configured and positioned to secure a connection between the urethral tube and the urethra and to prevent urine from leaking through that connection.
Example 11 is the implantable medical device according to any of Examples 1 to 10, in which the collection container includes flaps configured to be sutured to tissue of the body for fixation of the collection container.
Example 12 is the implantable medical device according to any of Examples 1 to 11, in which the pump is configured to exert a negative pressure that opens the unidirectional connection valve to allow the urine to flow from the collection container to the pump and closes the unidirectional urethral valve to prevent the urine from flowing from the pump to the urethra when the pump is not pressed.
Example 13 is the implantable medical device according to Example 12, in which the pump is configured to allow the patient to perceive a volume of the urine in the pump approaching a volume capacity of the pump and to exert a force that closes the unidirectional connection valve to prevent the urine from flowing from the collection container to the pump and opens the unidirectional urethral valve to allow the urine to flow from the pump to the urethra when the pump is pressed.
Example 14 is the implantable medical device according to any of Examples 1 to 13, in which the pump includes a hemispherical top portion and a flat base portion each having a wall, the base portion configured to be affixed to the fascia of the abdominal muscles, the wall of the flat base portion substantially thicker than the wall of the hemispherical top portion.
Example 15 is the implantable medical device according to any of Examples 1 to 14, further including: a cleaning port configured to be placed outside of the abdominal cavity and to receive a liquid cleaning agent; a cleaning tube connected between the cleaning port and the collection container to allow fluid communication from the cleaning port to the collection container; and a unidirectional cleaning valve coupled to the cleaning tube and configured to allow the liquid cleaning agent to flow from the cleaning port to the collection container.
Example 16 is the implantable medical device according to Example 15, in which the cleaning port is configured to be pierced by a hollow needle, to receive the liquid cleaning agent by injection through the hollow needle, and to self-seal upon removal of the hollow needle.
Example 17 is the implantable medical device according to any of Examples 1 to 16, in which the implantable medical device is substantially made of biocompatible elastic material.
Example 18 is the implantable medical device according to Example 17, in which the implantable medical device is entirely made of medical grade silicone.
Example 19 is the implantable medical device according to Example 17, in which the implantable medical device is substantially made of medical grade silicone.
Example 20 is a method for replacing functions of a urinary bladder using an implantable medical device placed in a body of a patient having kidneys, ureters, a urethra, an abdominal cavity, and a pelvic floor. The method can include: collecting urine from kidneys of the body using a collection container placed within the abdominal cavity through first and second ureteral tubes connected between the ureters and the collection container; preventing the collected urine from flowing back to the kidneys using first and second anti-reflux valves positioned in the collection container and coupled to the first and second ureteral tubes; allowing the urine to flow from the collection container to a pump subcutaneously placed outside of the abdominal cavity through a connection tube connected between the collection container and the pump when the pump is not pressed; and allowing the urine to flow from the pump to the urethra through a urethral tube connected between the pump and the urethra when the pump is pressed.
Example 21 is the method according to Example 20, further including maintaining an internal negative pressure in the collection container using a degree of resilience of the collection container to actively draw the urine from the kidneys to the collection container through the ureters, the first and second ureteral tubes, and the first and second anti-reflux valves and to prevent urinary stasis in the kidneys.
Example 22 is the method according to any of Examples 20 and 21, in which allowing the urine to flow from the pump to the urethra includes allowing the urine to flow from the pump to the urethra when a pressure applied on the pump exceeds a pressure level set using a high-pressure gradient valve positioned in the pump and coupled to the urethral tube.
Example 23 is the method according to any of Examples 20 to 22, further including securing connections each between a ureteral tube of the first and second ureteral tubes and a ureter of the ureters using two rings attached to an end portion of the ureteral tube inserted into the ureter and a suture passing through a wall of the ureter wall between the two ureteral rings and tied around the end portion of the ureteral tube.
Example 24 is the method according to any of Examples 20 to 23, further including securing a connection between the urethral tube and the urethra using mesh extensions affixed to the urethral tube and affixed to the pelvic floor.
Example 25 is the method according to Example 24, further including securing the connection between the urethral tube and the urethra using a urethral ring attached to the urethral tube.
Example 26 is the method according to any of Examples 20 to 25, further including constructing a substantial portion of the implantable medical device using medical grade silicone.
Example 27 is the method according to any of Examples 20 to 26, further including configuring the pump to allow the patient to perceive fullness of the pump as an indication of need for urination.
Example 28 is the method according to any of Examples 20 to 27, further including receiving a liquid cleaning agent into the collection container for cleaning interior space of the implantable medical device.
Example 29 is the method according to Example 28, in which receiving the liquid cleaning agent into the collection container includes: receiving the liquid cleaning agent from a cleaning port subcutaneously placed outside of the abdominal cavity and connected to the collection container through a cleaning tube; and receiving the liquid cleaning agent injected into the cleaning port using a hollow needle piercing the cleaning port.
Example 30 is the method according to any of Examples 28 and 29, in which receiving the liquid cleaning agent includes receiving one or more substances for disinfection and prevention of bladder stone formation.
Example 31 is an implantable medical device configured to be used as an artificial urinary bladder in a body having kidneys, ureters, a urethra, an abdominal cavity, and a pelvic floor. The implantable medical device can include: first means for being placed within the abdominal cavity and collecting urine produced by the kidneys; anti-reflux means for preventing the collected urine from flowing back to the kidneys; and second means for being placed subcutaneously outside of the abdominal cavity, receiving the collected urine, and expelling the received urine from the body when being pressed.
Example 32 is the implantable medical device according to Example 31, in which the first means includes: first tubes configured to be connected to the ureters; and a first elastic container coupled to the first tubes and configured to receive the urine produced by the kidneys through the first tubes and the anti-reflux means.
Example 33 is the implantable medical device according to Example 32, in which the first elastic container is configured to actively draw the urine produced by the kidneys through the first tubes and the anti-reflux means.
Example 34 is the implantable medical device according to any of Examples 32 and 33, in which the anti-reflux means includes duckbill valves each coupled to a tube of the first tubes and positioned in the first elastic container.
Example 35 is the implantable medical device according to any of Examples 31 to 34, in which the second means includes: a second tube configured to be connected to the urethra; and a second elastic container coupled to the second tube and configured to receive the collected urine from the first means and to expel the received urine from the body when being pressed.
Example 36 is the implantable medical device according to Example 35, in which the second means further includes: means for allowing the urine to flow from the first means to the second means unidirectionally when a pressure applied on the second elastic container does not exceed a threshold pressure level; and means for allowing the urine to flow from the second means to the urethra unidirectionally when the pressure applied on the second elastic container exceeds the threshold pressure level.
Example 37 is the implantable medical device according to any of Examples 35 and 36, in which the second elastic container is configured to allow the body to perceive a volume of the urine in the second elastic container approaching a volume capacity of the second elastic container.
Example 38 is the implantable medical device according to any of Examples 35 to 37, in which the second means further includes mesh extensions affixed to the second tube and configured to be affixed to the pelvic floor.
Example 39 is the implantable medical device according to any of Examples 31 to 38, further including means for receiving a liquid cleaning agent into the implantable medical device for cleaning interior space of the implantable medical device.
Example 40 is the implantable medical device according to Example 39, in which the means for receiving the liquid cleaning agent into the implantable medical device includes means for being pierced by a hollow needle through which the liquid cleaning agent is to be injected into the implantable medical device.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. An implantable medical device for collecting and discharging urine from a body of a patient having kidneys, ureters, a urethra, an abdominal cavity, abdominal muscles having a fascia, and a pelvic floor, the implantable medical device comprising:
first and second ureteral tubes configured to be connected to the ureters to receive the urine from the kidneys through the ureters;
a collection container coupled to the first and second ureteral tubes and configured to be placed inside the abdominal cavity and to receive the urine from the first and second ureteral tubes;
first and second anti-reflux valves positioned in the collection container and respectively coupled to first and second ureteral tubes, the first and second anti-reflux valves configured to prevent urine from flowing from the collection container to the kidneys;
a pump configured to be placed outside of the abdominal cavity;
a connection tube connected between the collection container and the pump to provide for fluid communication from the collection container to the pump;
a unidirectional connection valve coupled to the connection tube and configured to allow the urine to flow from the collection container to the pump;
a urethral tube connected to the pump and configured to be connected to the urethra; and
a unidirectional urethral valve coupled to the urethral tube and configured to allow the urine to flow from the pump to the urethra.

2. The implantable medical device of claim 1, wherein the first and second anti-reflux valves each comprise a duckbill valve.

3. The implantable medical device of claim 1, wherein the collection container is configured to have a degree of resilience determined for maintaining an internal negative pressure to actively draw urine from the kidneys to the collection container through the ureters, the first and second ureteral tubes, and the first and second anti-reflux valves and to prevent urinary stasis in the kidneys.

4. The implantable medical device of claim 3, wherein the collection container has a wall, and a thickness of the wall is determined to provide the degree of resilience.

5. The implantable medical device of claim 1, wherein the urine is discharged from the body in response to a pressure applied on the pump exceeding a threshold pressure level, and the unidirectional urethral valve comprises a high-pressure gradient valve configured to have an opening pressure determined based on the threshold pressure level for allowing flow of the urine from the pump to the urethral tube.

6. The implantable medical device of claim 1, further comprising two ureteral rings attached to each ureteral tube of the first and second urethral tubes, the two ureteral rings configured and positioned to secure a connection between the each ureteral tube to a respective ureter of the ureters and to prevent the urine from leaking through that connection.

7. The implantable medical device of claim 1, further comprising extensions affixed to the urethral tube, the extensions configured and positioned to be affixed to the pelvic floor after the urethral tube is inserted into the urethra.

8. The implantable medical device of claim 7, further comprising a urethral ring attached to the urethral tube, the urethral ring configured and positioned to secure a connection between the urethral tube and the urethra and to prevent urine from leaking through that connection.

9. The implantable medical device of claim 1, further comprising:
a cleaning port configured to be placed outside of the abdominal cavity and to receive a liquid cleaning agent;
a cleaning tube connected between the cleaning port and the collection container to allow fluid communication from the cleaning port to the collection container; and
a unidirectional cleaning valve coupled to the cleaning tube and configured to allow the liquid cleaning agent to flow from the cleaning port to the collection container.

10. The implantable medical device of claim 1, wherein the implantable medical device is substantially made of medical grade silicone.

11. A method for replacing functions of a urinary bladder using an implantable medical device placed in a body of a patient having kidneys, ureters, a urethra, an abdominal cavity, and a pelvic floor, the method comprising:
collecting urine from kidneys of the body using a collection container placed within the abdominal cavity through first and second ureteral tubes connected between the ureters and the collection container;
preventing the collected urine from flowing back to the kidneys using first and second anti-reflux valves positioned in the collection container and coupled to the first and second ureteral tubes;
allowing the urine to flow from the collection container to a pump subcutaneously placed outside of the abdominal cavity through a connection tube connected between the collection container and the pump when the pump is not pressed; and
allowing the urine to flow from the pump to the urethra through a urethral tube connected between the pump and the urethra when the pump is pressed.

12. The method of claim 11, further comprising maintaining an internal negative pressure in the collection container using a degree of resilience of the collection container to actively draw the urine from the kidneys to the collection container through the ureters, the first and second ureteral tubes, and the first and second anti-reflux valves and to prevent urinary stasis in the kidneys.

13. The method of claim 11, wherein allowing the urine to flow from the pump to the urethra comprises allowing the urine to flow from the pump to the urethra when a pressure applied on the pump exceeds a pressure level set using a high-pressure gradient valve positioned in the pump and coupled to the urethral tube.

14. The method of claim 11, further comprising securing connections each between a ureteral tube of the first and second ureteral tubes and a ureter of the ureters using two rings attached to an end portion of the ureteral tube inserted into the ureter and a suture passing through a wall of the ureter wall between the two ureteral rings and tied around the end portion of the ureteral tube.

15. The method of claim 11, further comprising securing a connection between the urethral tube and the urethra using mesh extensions affixed to the urethral tube and affixed to the pelvic floor.

16. The method of claim 15, further comprising securing the connection between the urethral tube and the urethra using a urethral ring attached to the urethral tube.

17. The method of claim 11, further comprising constructing a substantial portion of the implantable medical device using medical grade silicone.

18. The method of claim 11, further comprising configuring the pump to allow the patient to perceive fullness of the pump as an indication of need for urination.

19. The method of claim 11, further comprising receiving a liquid cleaning agent into the collection container for cleaning interior space of the implantable medical device, the liquid cleaning agent including one or more substances for at least one of disinfection or prevention of bladder stone formation.

20. The method of claim 19, wherein receiving the liquid cleaning agent into the collection container comprises:
receiving the liquid cleaning agent from a cleaning port subcutaneously placed outside of the abdominal cavity and connected to the collection container through a cleaning tube; and
receiving the liquid cleaning agent injected into the cleaning port using a hollow needle piercing the cleaning port.
